(19) United States
(12) Patent Application Publication
(10) Pub. No.: US 2024/0122494 A1
(43) Pub. Date: Apr. 18, 2024

(54) TECHNIQUES FOR ASSESSING CANCEROUS TISSUE DURING SURGICAL BIOPSIES

(71) Applicant: NOVASCAN, INC., Chicago, IL (US)

(72) Inventors: Les BOGDANOWICZ, Park Ridge, IL (US); Donato M. CERES, Chicago, IL (US); Onur FIDANER, Covington, LA (US); Alexander GRYCUK, Mt. Prospect, IL (US); Martina GUIDETTI, Chicago, IL (US); Daniel Steven GEHRKE, Chicago, IL (US); Margaret JOHN, Chicago, IL (US); Erik Michael KUNDRO, Chicago, IL (US); Federica DIBENNARDO, Chicago, IL (US); Constantine Bovalis, Chicago, IL (US)

(21) Appl. No.: 18/485,265

(22) Filed: Oct. 11, 2023

Related U.S. Application Data
(60) Provisional application No. 63/379,613, filed on Oct. 14, 2022.

(51) Int. Cl. A61B 5/0538; A61B 5/00

(52) U.S. Cl. CPC A61B 5/0538; A61B 5/742; A61B 2562/046

(57) ABSTRACT

In various embodiments, a medical device includes: a tissue sample-receiving surface; an electrode array that includes a first electrode and a second electrode that are positioned to contact a tissue sample disposed on the tissue sample-receiving surface; and an impedance bridge that is communicatively coupled to the electrode array.

### CROSS-REFERENCES TO RELATED APPLICATIONS

The present application claims the benefit of U.S. Provisional Patent Application No. 63/379,613, filed Oct. 14, 2022. The subject matter of this related application is hereby incorporated herein by reference.

### BACKGROUND

### Field of the Various Embodiments

Embodiments of the present disclosure relate generally to electronics and medical diagnostic technology and, more specifically, to techniques for assessing cancerous tissue during surgical biopsies.

### DESCRIPTION OF THE RELATED ART

In conventional practice, to provide an accurate cancer diagnosis of a suspect lesion, a physician takes one or more biopsy tissues samples from the suspect lesion with either a forceps device or needle device. The tissue samples are then sent for pathological analysis to an outside lab that may not provide the physician with a pathological assessment for multiple days or weeks. Typically, the physician takes multiple samples to increase the likelihood of capturing enough of the suspect tissue for an accurate diagnosis. However, there is nevertheless a relatively high likelihood that adequate sampling of the suspect lesion has not occurred, resulting in a non-diagnostic biopsy measurement that fails to indicate whether the lesion is cancerous or non-cancerous. For example, for numerous cancers, the rate of non-diagnostic biopsy measurements can be as high as 40%. The fact that suspect lesions are usually heterogeneous in nature and that suspect lesions also are typically not directly visible to the physician are two reasons why the percentage of non-diagnostic biopsy measurements is so high. Compounding these issues is the fact, noted above, that the physician usually has to wait days or weeks to receive the biopsy results.

Because a timely diagnosis of many cancers is critical for successful treatment, the ROSE (Rapid On-Site Evaluation) procedure has been developed to determine whether the quality of biopsy tissues samples is sufficient for subsequent pathological analysis. In the ROSE procedure, an onsite pathologist performs pathological analysis of biopsy tissues as they are taken from the patient by the physician, thereby providing more immediate feedback to the physician who is performing the biopsy. This procedure enables a physician to perform additional biopsies and/or change the region of patient anatomy being biopsied when non-diagnostic or non-cancerous measurements occur.

One drawback to the ROSE procedure is that the procedure can alter or consume a significant portion of each tissue sample tested. In that regard, a ROSE pathologist typically prepares slides of a tissue sample for microscopic evaluation by rubbing the tissue sample with the slide. Because the microscopic evaluation of the ROSE procedure benefits from more tissue on the slide, a substantial portion of a given tissue sample is oftentimes used to perform the procedure. As a result, the reliability of subsequent pathological analyses that are performed using the same given tissue sample can be adversely affected.

Another drawback to the ROSE procedure is that the procedure can be disruptive to the workflow of the physician who is performing the biopsy. While the ROSE procedure enables a biopsy physician to take additional samples in response to non-diagnostic tissue samples, the pathological analysis of each sample causes a delay on the order of ten minutes or more. Accordingly, the ROSE procedure can significantly interrupt a biopsy procedure, particularly if a large number of tissue samples are needed to complete the ROSE procedure. These types of interruptions can substantially increase the time needed to perform and complete a biopsy procedure, which increases the clinical resources needed to perform and complete the biopsy procedure and is also highly undesirable for the patient.

As the foregoing illustrates, what is needed in the art are more effective techniques for assessing tissue samples during surgical biopsies.

### SUMMARY

Embodiments are disclosed for medical devices. In various embodiments, a medical device includes: a tissue sample-receiving surface; an electrode array that includes a first electrode and a second electrode that are positioned to contact a tissue sample disposed on the tissue sample-receiving surface; and an impedance bridge that is communicatively coupled to the electrode array.

Embodiments are disclosed for analyzing a tissue sample. In various embodiments, a method includes recording, at one or more frequencies, one or more impedance measurements associated with an electrode array included in a medical device, wherein the electrode array includes a first electrode that contacts the tissue sample while disposed on a tissue sample-receiving surface and a second electrode that also contacts the tissue sample; determining, based on the one or more impedance measurements, a tissue type of the tissue sample; and causing an indicator of the tissue type to be displayed.

At least one technical advantage of the disclosed design and techniques relative to the prior art is that the disclosed design and techniques provide immediate, real-time feedback to a clinician who is performing a surgical biopsy. Consequently, when a tissue sample is determined to be non-diagnostic via the disclosed design and techniques, the clinician can immediately take one or more additional tissue samples, thereby avoiding the long delays in diagnosing non-diagnostic tissue samples that are typical with conventional approaches. In addition, because the disclosed design and techniques provide feedback to physicians within seconds, the workflow of a surgical biopsy procedure is not substantially impacted, and the duration of the surgical biopsy procedure is not significantly extended. A further technical advantage of the disclosed techniques is that tissue samples taken by a clinician are not altered; therefore, the reliability of subsequent pathological analyses are not adversely impacted. These technical advantages provide one or more technological advancements over prior art approaches.

### BRIEF DESCRIPTION OF THE DRAWINGS

FIG. 1 illustrates a medical device configured to implement one or more aspects of the various embodiments.

FIG. 2 is a more detailed illustration of the medical device of FIG. 1, according to various embodiments.

FIG. 3 is an exploded view of the sample module adapter of FIG. 1, according to various embodiments.

FIG. 4 illustrates a biopsy tissue sample being placed onto the biopsy sample strainer of FIG. 3, according to various embodiments.

FIG. 5A is a more detailed illustration of the sample-facing surface of the medallion electrode array of FIG. 3, according to various embodiments.

FIG. 5B is a more detailed illustration of the reverse surface of the medallion electrode array 320 of FIG. 3, according to various embodiments.

FIG. 6 schematically illustrates a tab electrode that can be employed to contact a biopsy tissue sample, according to various embodiments.

FIG. 7 illustrates a biopsy tissue sample being placed onto sample-facing surface of the ab electrode of FIG. 6, according to various embodiments.

FIG. 8 illustrates the medical device of FIG. 1 juxtaposed with a first sample module adapter and a second sample module adapter, according to various embodiments.

FIG. 9 schematically illustrates an array of transparent electrodes that can be employed to contact a biopsy tissue sample, according to various embodiments.

FIG. 10 is a flow diagram of performing a biopsy procedure on a tissue sample, according to various embodiments.

For clarity, identical reference numbers have been used, where applicable, to designate identical elements that are common between figures. It is contemplated that features of one embodiment may be incorporated in other embodiments without further recitation.

### DETAILED DESCRIPTION

In the following description, numerous specific details are set forth to provide a more thorough understanding of the various embodiments. However, in the range of embodiments of the concepts includes some embodiments omitting one or more of these specific details.

### System Overview

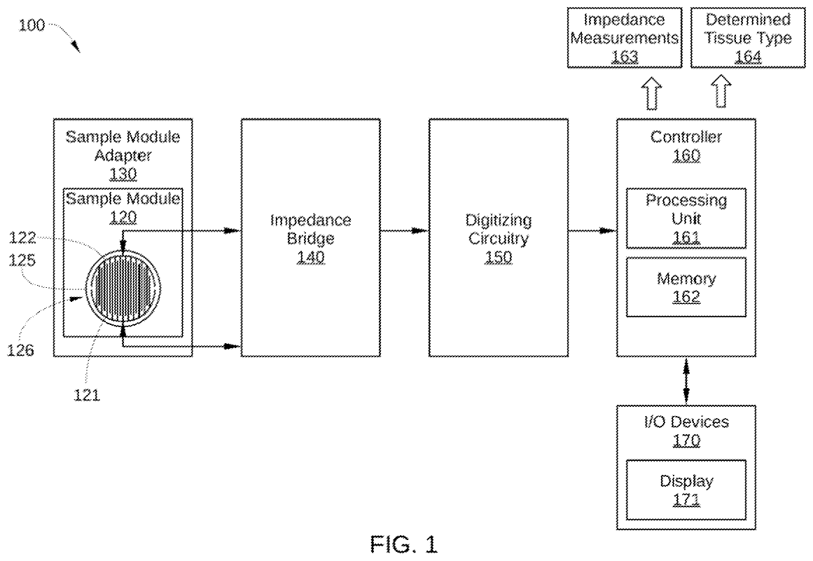

FIG. 1 illustrates a medical device 100 configured to implement one or more aspects of the various embodiments. Medical device 100 enables ex vivo real-time biopsy assessment of tissue samples, such as biopsy tissue sampled from a region of patient anatomy as part of a surgical biopsy procedure. Based on one or more bioimpedance biomarkers, medical device 100 determines a tissue type of a tissue sample, thereby providing immediate feedback to a clinician performing the surgical biopsy procedure. As described in greater detail below, medical device 100 determines a tissue type of a particular tissue sample based on impedance measurements of the tissue sample. As a result, the clinician can be informed in seconds that a specific sample includes cancerous tissue, non-cancerous tissue, or pre-cancerous tissue. Further, there is little or no disruption to the workflow of the surgical biopsy procedure. As shown, medical device 100 includes, without limitation, a sample module 120, a sample module adapter 130, an impedance bridge 140, digitizing circuitry 150, a controller 160, and input/output (I/O) devices 170.

Sample module 120 holds a tissue sample (not shown) and contacts the tissue sample with a first electrode 121 and a second electrode 122 of an electrode array 126, so that an impedance scan of the tissue sample can be performed. In the embodiment illustrated in FIG. 1, sample module 120 includes a tissue sample-receiving surface 125 and electrode array 126. For an impedance scan, a tissue sample is placed on tissue sample-receiving surface 125 and in contact with first electrode 121 and second electrode 122 of electrode array 126. Generally, sample module 120 is included in sample module adapter 130.

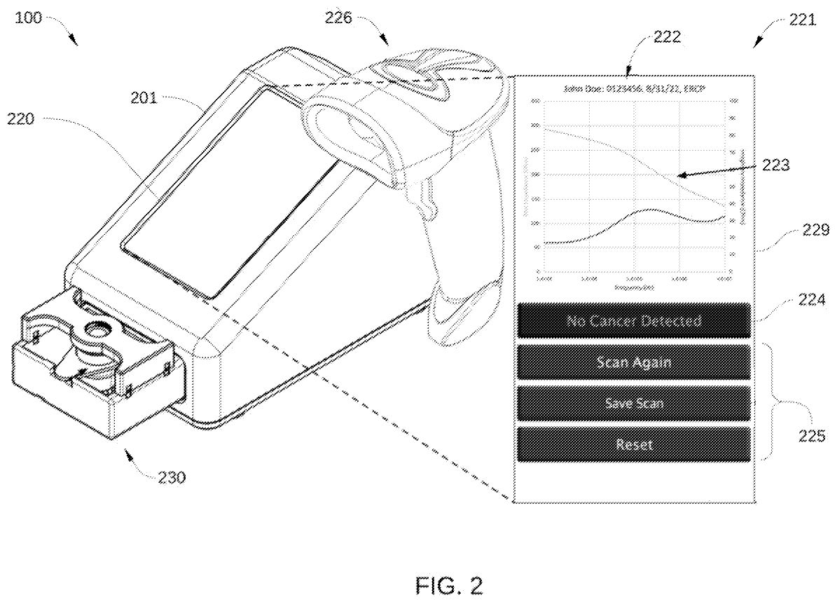

Sample module adapter 130 communicatively couples sample module 120 to impedance bridge 140. Thus, sample module adapter 130 electrically couples first electrode 121 and second electrode 122 to appropriate electrical contacts that are associated with impedance bridge 140. In some embodiments, multiple different configurations of sample module 120 can be employed with medical device 100. In such embodiments, a first sample module adapter 130 is configured to communicatively couple a first configuration of sample module 120 to impedance bridge 140, a second sample module adapter 130 is configured to communicatively couple a second configuration of sample module 120 to impedance bridge 140, and so on. In this way, medical device 100 can utilize multiple different configurations of sample module 120. Various embodiments of sample module 120 and sample module adapter 130 are described below in conjunction with FIGS. 2-9.

Impedance bridge 140 can be an impedance load that controller 160 measures to determine an impedance of a circuit that includes impedance bridge 140, an amplifier (when present), and electrode array 126. For example, in some embodiments, when first electrode 121 and second electrode 122 of electrode array 126 contact a tissue sample on sample-receiving surface 125, controller 160 generates frequencies for a current conducted within the circuit that includes impedance bridge 140 and electrode array 126. In various embodiments, impedance bridge 140 further includes an amplifier (not shown) that is an analog interface amplifier for amplifying a supplied voltage and/or a return voltage while a current is conducted at various frequencies between impedance bridge 140 and the electrode array 126. It is noted that impedance bridge 140 and electrode array 126 form a circuit when a tissue sample contacts first electrode 121 and second electrode 122 of electrode array 126, sample module 120 is disposed in sample module adapter 130, and sample module adapter 130 is coupled to medical device 100. Digitizing circuitry 150 digitizes signals in such a circuit to enable operation of controller 160.

Controller 160 includes a processing unit 161 and a memory 162 and performs one or more operations to implement various embodiments described herein. For example, in some embodiments, controller 160 executes a program stored in memory 162 to conduct multiple specific electrical measurements on a tissue sample using first electrode 121 and second electrode 122 of electrode array 126. In some embodiments, controller 160 may receive instructions from a user via I/O devices 170 to perform the electrical measurements, such as one or more impedance measurements 163, using electrode array 126. In some embodiments, controller 160 may receive instructions from the user via I/O devices 170 to store data, such as data associated with one or more impedance measurements 163. In some embodiments, the stored data associated with impedance measurements 163 includes the measured electrical properties determined by the measurement circuit that includes impedance bridge 140 and electrode array 126. For example, in some embodiments, controller 160 stores the measured voltage and the measured current for an input signal at a specific operating frequency.

In some embodiments, controller 160 determines a determined tissue type 164 of a tissue sample contacting first electrode 121 and second electrode 122. In the embodiments, the determined tissue type is based on one or more impedance measurements 163 associated with the sample contacting first electrode 121 and second electrode 122. In various embodiments, controller 160 determines a determined tissue type 164 by comparing impedance measurements 163 with one or more characteristic impedance measurements associated with one or more tissue types. For example, and without limitation, based on the comparing, controller 160 can determine which tissue type is associated with characteristic impedance measurements that are closest to impedance measurements 163 of the sample tissue contacting first electrode 121 and second electrode 122.

In various embodiments, controller 160 can determine a Cole relaxation frequency of the tissue sample based on impedance measurements 163. Specifically, controller 160 computes the Cole relaxation frequency for the tissue sample based on the computed impedances corresponding to the operating frequency, where the Cole relaxation frequency for the tissue sample reflects the rate at which a cell membrane discharges a stored electrical charge. In the embodiments, Controller 160 compares the Cole relaxation frequency to one or more characteristic Cole relaxation frequencies of one or more tissue types. The Cole relaxation frequency for a particular tissue sample corresponds to a frequency associated with a greatest impedance measurement 163 included in the one or more impedance measurements 163 for that particular tissue sample. In various embodiments, the Cole relaxation frequency is a frequency of a maximum normalized impedance measurement of the tissue sample contacting first electrode 121 and second electrode 122.

Due to the contrasting electrical properties of malignant cells and non-malignant cells, malignant cells have a Cole relaxation frequency that is over one thousand times smaller than the Cole relaxation frequency of a non-malignant cell. Consequently, in some embodiments, based on a Cole relaxation frequency below a lower threshold frequency (e.g., 105 Hz), controller 160 can determine that the tissue sample contacting first electrode 121 and second electrode 122 is a non-tumor tissue type (also referred to as non-cancerous tissue). Similarly, in some embodiments, based on a Cole relaxation frequency above an upper threshold frequency, controller 160 can determine that the tissue sample contacting first electrode 121 and second electrode 122 is a tumor tissue type (also referred to as cancerous tissue). Further, for example and without limitation, based on a Cole relaxation frequency that is between the lower threshold frequency (e.g., of 500 kHz) and above the upper threshold frequency (e.g., of 1 MHz), controller 160 can determine that the tissue sample contacting first electrode 121 and second electrode 122 is a precancerous tissue.

In some embodiments, processing unit 161 can be a single central processing unit (CPU), or combination of processing units. Processing unit 161 may be any technically-feasible hardware unit capable of processing data and/or executing software code. In some embodiments, processing unit 161 receives instructions from a user via I/O devices 170 and/or from memory 162 and may execute such instructions. In some embodiments, processing unit 161 implements one or more techniques executed by controller 160. In some embodiments, memory 162 is configured to store data and/or software applications. Memory 162 may include a random access memory (RAM) module, hard disk, flash memory unit, or any other type of memory unit or combination thereof. Controller 160 and I/O units 130 are configured to read data from memory 162 and to write data to memory 162.

Input/output (I/O) devices 170 may include devices capable of receiving one or more inputs, including a keyboard, mouse, input tablet, camera, and/or three-dimensional (3D) scanner. In some embodiments, I/O devices 170 may also include devices capable of providing one or more outputs, such as a speaker, printer, or a display 171. Display 171 displays data generated by medical device 100. In an embodiment, display 171 displays one or more of the computed Cole relaxation frequency and/or a determined tissue type 164. In some embodiments, display 171 may refresh the data generated by medical device 100 while medical device 100 performs measurements on a particular tissue sample. I/O devices 170 may also include devices capable of both receiving inputs and providing outputs, such as a touchscreen and a universal serial bus (USB) port.

FIG. 2 is a more detailed illustration of medical device 100, according to various embodiments. As shown, medical device 100 includes, without limitation, a table-top housing 201, a display 220, and a sample module adapter 230. Also shown in FIG. 2 is a display output 221, which includes a graphical user interface 229. In the embodiment illustrated in FIG. 2, graphical user interface 229 includes, without limitation, patient-specific information 222 for a patient associated with a tissue sample, a graphical representation 223 of the results of a scan of a tissue sample, textual results 224 of a scan of the tissue sample, and one or more interaction buttons 225 for receiving user inputs. In the embodiment illustrated in FIG. 2, medical device 100 includes a bar code scanner 226 for inputting patient information.

### Medallion Electrode Embodiment

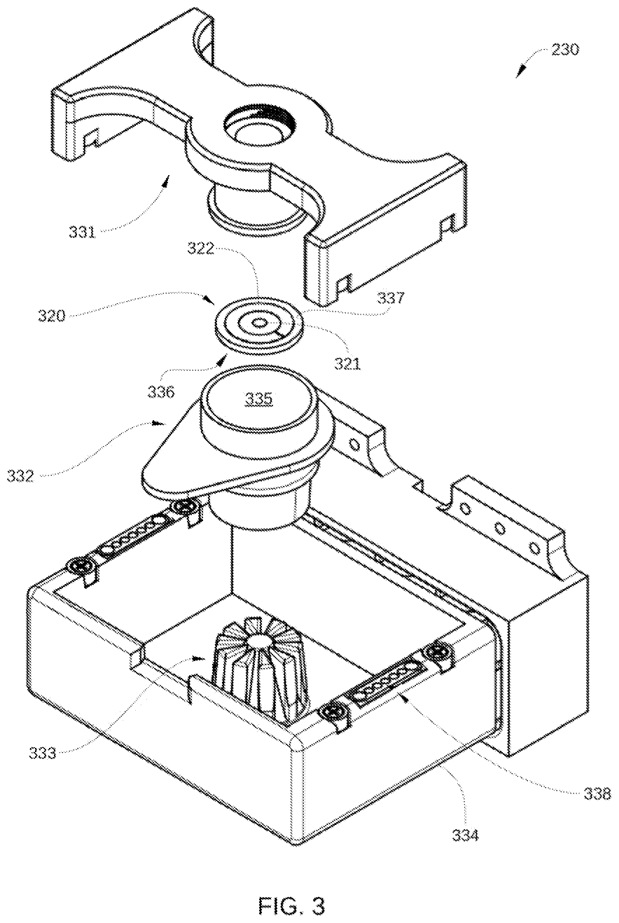

FIG. 3 is an exploded view of sample module adapter 230, according to various embodiments. Sample module adapter 230 is configured to position a medallion electrode array 320 so that a tissue sample (not shown) contacts a first electrode 321 electrode and a second electrode 322 of medallion electrode array 320. Sample module adapter 230 is further configured to communicatively couple medallion electrode array 320 and the tissue sample to impedance bridge 140 in FIG. 1. As shown, sample module adapter 230 includes, without limitation, a compressive tissue sample holder 331, medallion electrode array 320, a biopsy sample strainer 332, a strainer alignment feature 333 for positioning biopsy sample strainer 332, electrical contacts 333, and a housing 334.

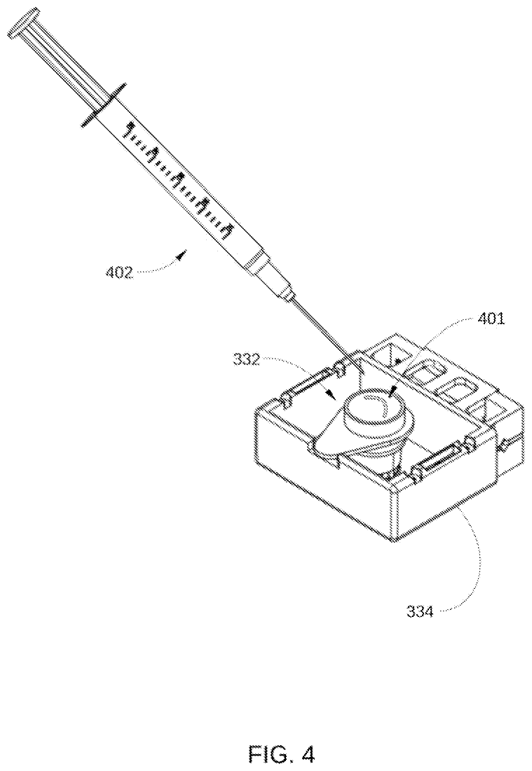

Compressive tissue sample holder 331, when installed over medallion electrode array 320 onto housing 334, presses medallion electrode array 320 against a tissue sample (not shown) that is disposed on a tissue sample-receiving surface 335 of sample module adapter 230. In the embodiment illustrated in FIG. 3, tissue sample-receiving surface 335 is a top surface of biopsy sample strainer 332, which in operation is coupled to strainer alignment feature 333. Compressive tissue sample holder 331 includes electrical conductors (not shown) that electrically couple medallion electrode array 320 to impedance bridge 140 of FIG. 1, for example via electrical contacts 333. Biopsy sample strainer 332 is positioned within medallion electrode array 320 via strainer alignment feature 333. In operation, a tissue sample is flushed onto biopsy sample strainer 332, and first electrode 321 and second electrode 322 contact the tissue sample when compressive tissue sample holder 331 is coupled to housing 334 of medallion electrode array 320. The placing of a biopsy tissue sample onto biopsy sample strainer 332 is shown in FIG. 4.

FIG. 4 illustrates a biopsy tissue sample 401 being placed onto biopsy sample strainer 332, according to various embodiments. Biopsy tissue sample 401 can be collected with a needle biopsy syringe 402 and placed on biopsy sample strainer 332 after collection as shown. Compressive tissue sample holder 331 (not shown in FIG. 4) is removed from housing 334 so that tissue sample-receiving surface 335 of biopsy sample strainer 332 is accessible.

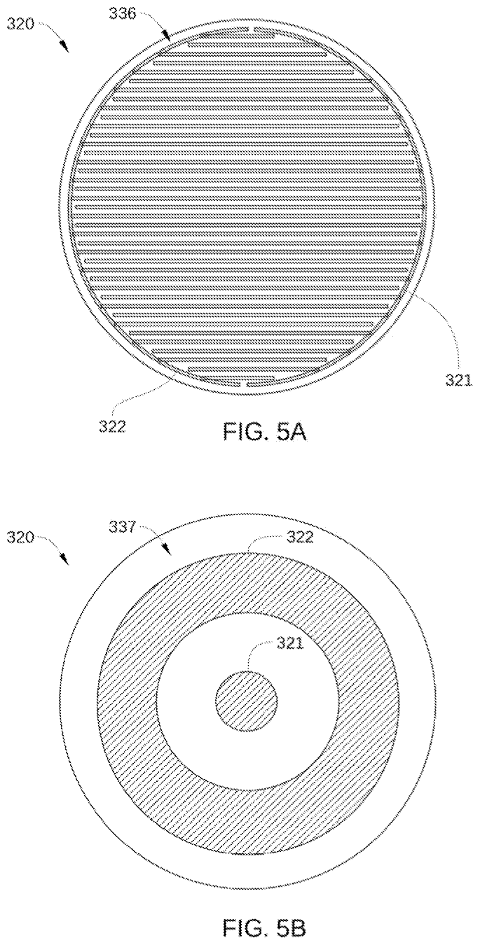

Returning to FIG. 3, medallion electrode array 320 includes first electrode 321 electrode and second electrode 322. In the embodiment illustrated in FIG. 3, a portion of first electrode 321 is disposed on a sample-facing surface 336 (not visible in FIG. 3) of medallion electrode array 320 and a portion of first electrode 321 is disposed on a reverse surface 337 of medallion electrode array 320. Similarly, a portion of second electrode 322 is disposed on sample-facing surface 336 and a portion of second electrode 322 is disposed on reverse surface 337. An embodiment of medallion electrode array 320 is described in greater detail below in conjunction with FIGS. 5A and 5B.

FIG. 5A is a plan view of sample-facing surface 336 of medallion electrode array 320, according to various embodiments, and FIG. 5B is a plan view of reverse surface 337 of medallion electrode array 320, according to various embodiments. As shown, a portion of first electrode 321 is disposed on sample-facing surface 336 and a portion (cross-hatched) of first electrode 321 is also disposed on reverse surface 337 of medallion electrode array 320. Similarly, a portion of second electrode 322 is disposed on sample-facing surface 336 and a portion (cross-hatched) of second electrode 322 is also disposed on reverse surface 337 of medallion electrode array 320. To facilitate sufficient electrical contact with a tissue sample disposed on tissue sample-receiving surface 335 of sample module adapter 230 (shown in FIG. 3), the portion of first electrode 321 disposed on sample-facing surface 336 includes a plurality of electrode elements and the portion of second electrode 322 disposed on sample-facing surface 336 includes a plurality of electrode elements. In the embodiment illustrated in FIG. 5A, the electrode elements included in first electrode 321 and disposed on sample-facing surface 336 are interdigitated with the elements included in second electrode 322 and disposed on sample-facing surface 336.

In some embodiments, to enable medallion electrode array 320 to be arranged in any rotational orientation, medallion electrode array 320 is round in configuration. Further, the portion of first electrode 321 disposed on reverse surface 337 is radially displaced from the portion of second electrode 322 disposed on reverse surface 337. Thus, medallion electrode array 320 can be electrically connected to conductors included in compressive tissue sample holder 331 regardless of the rotational orientation of medallion electrode array 320. For example, in the embodiment illustrated in FIG. 5B, the portion of first electrode 321 disposed on reverse surface 337 is located at a center location of reverse surface 337 and the portion of second electrode 321 is formed as a ring that is disposed radially outward on reverse surface 337 from the portion of first electrode 321.

### Tab Electrode Embodiment

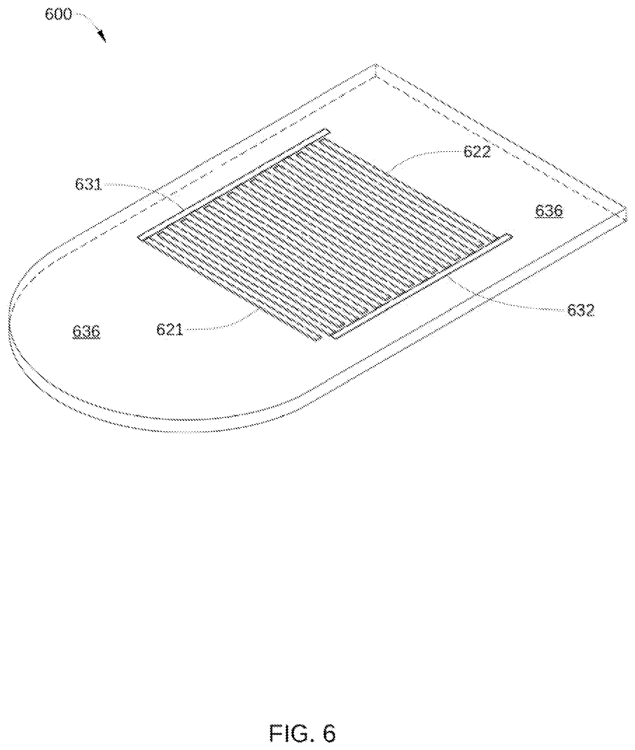

In some embodiments, impedance measurements of a tissue sample are collected via a tab electrode. In such embodiments, a tab electrode is employed to electrically contact the tissue sample instead of a medallion electrode. One such embodiment is described below in conjunction with FIG. 6.

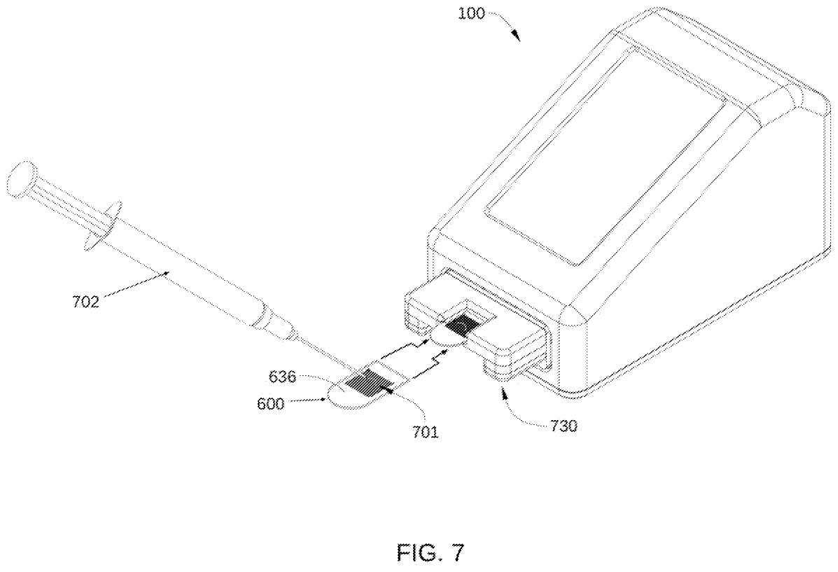

FIG. 6 schematically illustrates a tab electrode 600 that can be employed to contact a biopsy tissue sample (not shown), according to various embodiments. As shown, tab electrode 600 includes a first electrode 621 and a second electrode 622. First electrode 621 includes a plurality of electrode elements that are electrically coupled to each other via a first edge electrode element 631, and second electrode 622 includes a plurality of electrode elements that are electrically coupled to each other via a second edge electrode element 632. Similar to medallion electrode array 320 in FIG. 3, in the embodiment illustrated in FIG. 6, the electrode elements included in first electrode 621 are interdigitated with the plurality of electrode elements included in second electrode 622. Unlike medallion electrode array 320, the electrode elements included in first electrode 621 and the electrode elements included in second electrode 622 are formed on a sample-facing surface 636. Thus, in operation, when a tissue sample (not shown) is placed onto sample-facing surface 636, first electrode 621 and second electrode 622 contact the tissue sample. The placing of a biopsy tissue sample onto sample-facing surface 636 is shown in FIG. 7.

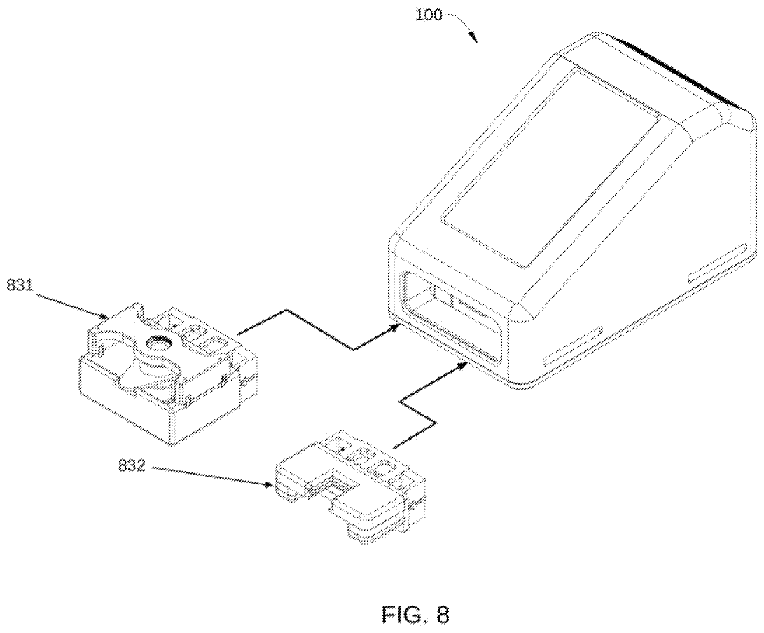

FIG. 7 illustrates a biopsy tissue sample 701 being placed onto sample-facing surface 636 of tab electrode 600, according to various embodiments. Biopsy tissue sample 701 can be collected with a needle biopsy syringe 702 and placed on sample-facing surface 636 after collection as shown. Also shown in FIG. 7 is tab electrode 600 coupled to a sample module adapter 730, which is configured for receiving tab electrode 600 and is shown coupled to medical device 100 in FIG. 7. In some embodiments, medical device 100 is configured to couple to multiple different configurations of sample module adapters, such as sample module adapter 230 of FIG. 2 and sample module adapter 730 of FIG. 7. One such embodiment is described below in conjunction with FIG. 8.

FIG. 8 illustrates medical device 100 juxtaposed with a first sample module adapter 831 and a second sample module adapter 832, according to various embodiments. In some embodiments, first sample module adapter 831 is consistent with tissue module adapter 230 of FIG. 2 and second sample module adapter 832 is consistent with sample module adapter 730 of FIG. 7. As shown, first sample module adapter 831 and second sample module adapter 832 can both be coupled to medical device 100. Thus, medical device 100 can employ either a medallion electrode or a tab electrode for contacting a tissue sample and taking impedance measurements.

### Transparent Electrode Embodiment

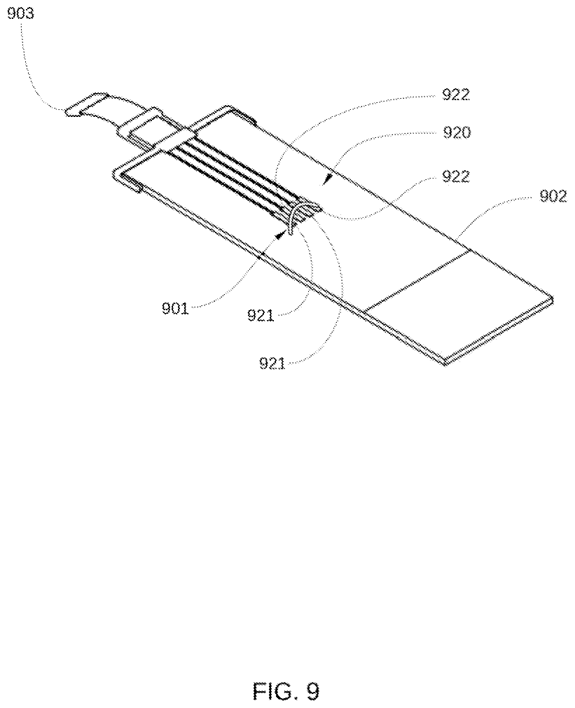

In some embodiments, impedance measurements of a tissue sample are collected via an array of transparent electrodes disposed on an optically transparent substrate. In such embodiments, the optically transparent substrate can be employed as an microscope slide. One such embodiment is described below in conjunction with FIG. 9.

FIG. 9 schematically illustrates an array 920 of transparent electrodes that can be employed to contact a biopsy tissue sample 901, according to various embodiments. Array 920 includes a first electrode 921 and a second electrode 922 and is formed or deposited on a transparent substrate 902. As shown, array 920 further includes an electrical connection 903 to impedance bridge 140 of FIG. 1.

In the embodiment illustrated in FIG. 9, first electrode 921 includes two transparent electrode elements that are electrically coupled to each other via a first common electrode element (not shown for clarity) and second electrode 922 includes two transparent electrode elements that are electrically coupled to each other via a second common electrode element (not shown for clarity). In some embodiments, the transparent electrode elements of first electrode 921 and second electrode 922 include one or more electrically conductive transparent materials, such as titanium nitride and/or indium tin oxide. In such embodiments, because first electrode 921 and second electrode 922 are substantially transparent to visible light, transparent substrate 902 can be employed as an optical microscope slide. Thus, impedance measurements of biopsy sample 901 can be performed when biopsy tissue sample 901 is disposed on transparent substrate 902. Then, because first electrode 921 and second electrode 922 do not interfere with microscope-based pathological assessment of biopsy tissue sample 901, conventional microscope-based pathological assessment of biopsy tissue sample 901 can be performed without removing biopsy tissue sample 901 from transparent substrate 902.

### Method for Assessing Cancerous Tissue During Surgical Biopsies

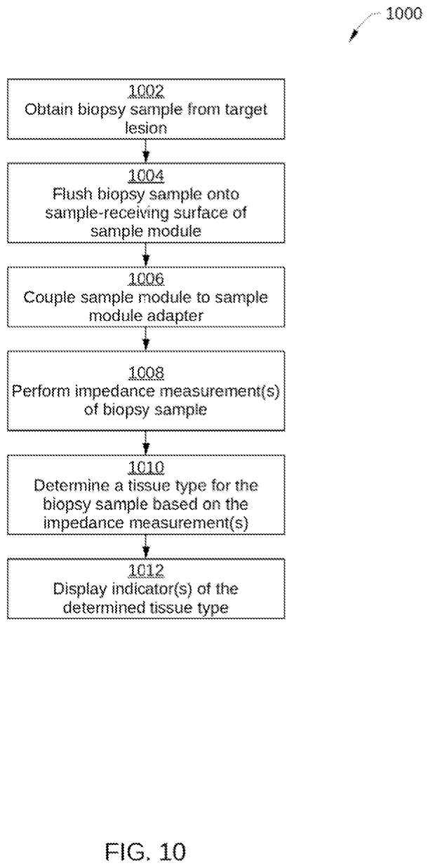

FIG. 10 is a flow diagram of performing a biopsy procedure on a tissue sample, according to various embodiments. Although the method steps are described in conjunction with the systems of FIGS. 1-8, persons skilled in the art will understand that any system configured to perform the method steps, in any order, falls within the scope of the present invention.

As shown, a method 1000 begins at step 1002, where a biopsy sample is obtained from a region of patient anatomy, such as a target lesion. In some instances, the lesion is imaged so that the location of the target lesion can be determined. For example, in some instances, the region of patient anatomy can be imaged via X-ray, ultra-sound, and/or magnetic resonance imaging (MRI). The biopsy sample is then taken using an appropriate biopsy procedure, where the appropriate biopsy procedure may be determined based on the location of the target lesion and/or the general region of patient anatomy of concern. Examples of such biopsy procedures include needle biopsy, endoscopic biopsy, skin biopsy, bone marrow biopsy, surgical biopsy, and the like. Typically, a needle or hollow tube is employed to take a particular biopsy sample from the region of anatomy or target lesion.

In step 1004, the biopsy sample is placed onto sample-receiving surface 125 of a sample module 120. For example, in some instances, the biopsy sample, such as a biopsy core from a needle biopsy syringe, is flushed with saline onto tissue sample-receiving surface 335 of sample module adapter 230. Alternatively, the biopsy sample can be flushed onto sample-facing surface 636 of a tab electrode 600 or onto a transparent substrate 902.

In step 1006, the sample module 120 on which the biopsy core has been placed is coupled to an appropriate sample module adapter 130, such as sample module adapter 230 or sample module adapter 730. In some embodiments, a removable compressive tissue sample holder is first used to press an electrode array of sample module 120 against the biopsy sample disposed on the tissue sample-receiving surface 125 of sample module 120.

In step 1008, one or more impedance measurement(s) 163 of the biopsy sample are performed with medical device 100. As described previously, one or more impedance measurements 163, at one or more frequencies, are performed and recorded via controller 160, impedance bridge 140, and electrode array 126.

In step 1010, controller 160 determines a determined tissue type 164 for the biopsy sample based on the impedance measurement(s) 163 performed in step 1008. In some embodiments, the determined tissue type 164 is further determined based on comparing the Cole relaxation frequency of the biopsy sample to one or more characteristic Cole relaxation frequencies of one or more tissue types. In such embodiments, the comparison can be based on a single threshold frequency or on a lower threshold frequency and an upper threshold frequency.

In embodiments in which the comparison is based on a single threshold frequency, controller 160 determines that the determined tissue type 164 is non-cancerous when the Cole relaxation frequency of the biopsy sample is less than the single threshold frequency and cancerous when the Cole relaxation frequency of the biopsy sample is greater than the single threshold frequency. Alternatively, in embodiments in which the comparison is based on a lower threshold frequency and an upper threshold frequency, the controller determines that the determined tissue type 164 is non-cancerous when the Cole relaxation frequency of the biopsy sample is less than the lower threshold frequency, cancerous when the Cole relaxation frequency of the biopsy sample is greater than the upper threshold frequency, and pre-cancerous when the Cole relaxation frequency of the biopsy sample is both greater than the lower threshold frequency and less than the upper threshold frequency. It is noted that threshold frequencies generally vary based on a particular tissue type, and can be determined empirically. For example, the threshold frequencies for pancreatic tissue can be significantly different that the threshold frequencies for bone marrow tissue, skin tissue, lung tissue, and the like.

In step 1012, controller 160 causes one or more indicators of determined tissue type 164 to be displayed. For example, in some embodiments, controller 160 causes graphical representation 223 of results for the biopsy sample to be displayed by display 171. Alternatively or additionally, in some embodiments, controller 160 causes textual results 224 of results for the biopsy sample to be displayed by display 171.

In sum, embodiments described herein enable the determination of a tissue type for a tissue sample. Based on ex vivo impedance measurements performed on the tissue sample, a medical device can determine whether the tissue sample is cancerous, non-cancerous, or pre-cancerous. An impedance bridge is employed to perform the impedance measurements without altering or consuming a the tissue sample, and an electrode array of the medical device is configured with interdigitated electrode elements to facilitate electrical contact with the tissue sample during such measurements.

At least one technical advantage of the disclosed design and techniques relative to the prior art is that the disclosed design and techniques provide immediate, real-time feedback to a clinician who is performing a surgical biopsy. Consequently, when a tissue sample is determined to be non-diagnostic via the disclosed design and techniques, the clinician can immediately take one or more additional tissue samples, thereby avoiding the long delays in diagnosing non-diagnostic tissue samples that are typical with conventional approaches. In addition, because the disclosed design and techniques provide feedback to physicians within seconds, the workflow of a surgical biopsy procedure is not substantially impacted, and the duration of the surgical biopsy procedure is not significantly extended. A further technical advantage of the disclosed techniques is that tissue samples taken by a clinician are not altered; therefore, the reliability of subsequent pathological analyses are not adversely impacted. These technical advantages provide one or more technological advancements over prior art approaches.
1. In an embodiment, a medical device, includes: a tissue sample-receiving surface; an electrode array that includes a first electrode and a second electrode that are positioned to contact a tissue sample disposed on the tissue sample-receiving surface; and an impedance bridge that is communicatively coupled to the electrode array.
2. The medical device of clause 1, wherein the first electrode includes a first plurality of electrode elements, and the second electrode includes a second plurality of electrode elements.
3. The medical device of clauses 1 or 2, wherein electrode elements of the first plurality of electrode elements are interdigitated between electrode elements of the second plurality of electrode elements.
4. The medical device of any of clauses 1-3, wherein the tissue sample-receiving surface is disposed on an optically transparent substrate.
5. The medical device of any of clauses 1-4, wherein the tissue sample-receiving surface is disposed on an optically transparent substrate and the electrode array is disposed on the tissue sample-receiving surface.
6. The medical device of any of clauses 1-5, wherein the first electrode and the second electrode are transparent to visible light.
7. The medical device of any of clauses 1-6, wherein the electrode array is disposed on the tissue sample-receiving surface.
8. The medical device of any of clauses 1-7, further comprising a removable a compressive tissue sample holder that presses the electrode array against the tissue sample disposed on the tissue sample-receiving surface.
9. The medical device of any of clauses 1-8, wherein the removable compressive tissue sample holder includes one or more electrical conductors that communicatively couple the impedance bridge and the electrode array.
10. The medical device of any of clauses 1-9, further comprising an adapter opening that selectively receives a first sample module adapter for containing the tissue sample or a second sample module adapter for containing the tissue sample.
11. The medical device of any of clauses 1-10, wherein the first sample module adapter includes the tissue sample-receiving surface and a removable compressive tissue sample holder that presses the electrode array against the tissue sample disposed on the tissue sample-receiving surface.
12. The medical device of any of clauses 1-11, wherein the second sample module adapter includes the tissue sample-receiving surface, and the electrode array is disposed on the tissue sample-receiving surface.
13. The medical device of any of clauses 1-12, further comprising a controller that, during operation, performs the steps of: recording, at one or more frequencies, one or more impedance measurements associated with the electrode array when the first electrode and the second electrode contact the tissue sample; determining, based on the one or more impedance measurements, a tissue type of the tissue sample; and causing an indicator of the tissue type to be displayed.
14. The medical device of any of clauses 1-13, wherein determining the tissue type comprises comparing the one or more impedance measurements to one or more characteristic impedance measurements associated with one or more tissue types.
15. In some embodiments, a method for analyzing a tissue sample includes: recording, at one or more frequencies, one or more impedance measurements associated with an electrode array included in a medical device, wherein the electrode array includes a first electrode that contacts the tissue sample while disposed on a tissue sample-receiving surface and a second electrode that also contacts the tissue sample; determining, based on the one or more impedance measurements, a tissue type of the tissue sample; and causing an indicator of the tissue type to be displayed.
16. The method of clause 15, wherein the tissue type is selected from the group consisting of cancerous tissue, non-cancerous tissue, and pre-cancerous tissue.
17. The method of clauses 15 or 16, wherein determining the tissue type comprises comparing the one or more impedance measurements to one or more characteristic impedance measurements associated with one or more tissue types.
18. The method of any of clauses 15-17, wherein determining the tissue type comprises: determining a Cole relaxation frequency of the portion of tissue based on the one or more impedance measurements, and comparing the Cole relaxation frequency of the portion of tissue to one or more characteristic Cole relaxation frequencies of one or more tissue types.
19. The method of any of clauses 15-18, wherein the Cole relaxation frequency corresponds to a frequency associated with a greatest impedance measurement included in the one or more impedance measurements.
20. The method of any of clauses 15-19, wherein the indicator is displayed on a display device associated with the medical device.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A medical device, comprising:
a tissue sample-receiving surface;
an electrode array that includes a first electrode and a second electrode that are positioned to contact a tissue sample disposed on the tissue sample-receiving surface; and
an impedance bridge that is communicatively coupled to the electrode array.

2. The medical device of claim 1, wherein the first electrode includes a first plurality of electrode elements, and the second electrode includes a second plurality of electrode elements.

3. The medical device of claim 2, wherein electrode elements of the first plurality of electrode elements are interdigitated between electrode elements of the second plurality of electrode elements.

4. The medical device of claim 1, wherein the tissue sample-receiving surface is disposed on an optically transparent substrate.

5. The medical device of claim 1, wherein the tissue sample-receiving surface is disposed on an optically transparent substrate and the electrode array is disposed on the tissue sample-receiving surface.

6. The medical device of claim 5, wherein the first electrode and the second electrode are transparent to visible light.

7. The medical device of claim 1, wherein the electrode array is disposed on the tissue sample-receiving surface.

8. The medical device of claim 1, further comprising a removable a compressive tissue sample holder that presses the electrode array against the tissue sample disposed on the tissue sample-receiving surface.

9. The medical device of claim 8, wherein the removable compressive tissue sample holder includes one or more electrical conductors that communicatively couple the impedance bridge and the electrode array.

10. The medical device of claim 1, further comprising an adapter opening that selectively receives a first sample module adapter for containing the tissue sample or a second sample module adapter for containing the tissue sample.

11. The medical device of claim 10, wherein the first sample module adapter includes the tissue sample-receiving surface and a removable compressive tissue sample holder that presses the electrode array against the tissue sample disposed on the tissue sample-receiving surface.

12. The medical device of claim 10, wherein the second sample module adapter includes the tissue sample-receiving surface, and the electrode array is disposed on the tissue sample-receiving surface.

13. The medical device of claim 1, further comprising a controller that, during operation, performs the steps of:
recording, at one or more frequencies, one or more impedance measurements associated with the electrode array when the first electrode and the second electrode contact the tissue sample;
determining, based on the one or more impedance measurements, a tissue type of the tissue sample; and
causing an indicator of the tissue type to be displayed.

14. The medical device of claim 1, wherein determining the tissue type comprises comparing the one or more impedance measurements to one or more characteristic impedance measurements associated with one or more tissue types.

15. A method for analyzing a tissue sample, the method comprising:
recording, at one or more frequencies, one or more impedance measurements associated with an electrode array included in a medical device, wherein the electrode array includes a first electrode that contacts the tissue sample while disposed on a tissue sample-receiving surface and a second electrode that also contacts the tissue sample;
determining, based on the one or more impedance measurements, a tissue type of the tissue sample; and
causing an indicator of the tissue type to be displayed.

16. The method of claim 15, wherein the tissue type is selected from the group consisting of cancerous tissue, non-cancerous tissue, and pre-cancerous tissue.

17. The method of claim 15, wherein determining the tissue type comprises comparing the one or more impedance measurements to one or more characteristic impedance measurements associated with one or more tissue types.

18. The method of claim 15, wherein determining the tissue type comprises:
determining a Cole relaxation frequency of the portion of tissue based on the one or more impedance measurements, and
comparing the Cole relaxation frequency of the portion of tissue to one or more characteristic Cole relaxation frequencies of one or more tissue types.

19. The method of claim 18, wherein the Cole relaxation frequency corresponds to a frequency associated with a greatest impedance measurement included in the one or more impedance measurements.

20. The method of claim 15, wherein the indicator is displayed on a display device associated with the medical device.
